(19) United States
(12) Patent Application Publication
(10) Pub. No.: US 2012/0253267 A1
(43) Pub. Date: Oct. 4, 2012

(54) COMBINED OTIC AND MEDICATION DISPENSER AND METHOD FOR TREATING OTIC INFECTIONS

(76) Inventors: DON C. REED, RANDOLPH, NJ (US); SEAMUS O'LOAN, WAYNE, PA (US)

(21) Appl. No.: 13/429,087

(22) Filed: Mar. 23, 2012

Related U.S. Application Data
(63) Continuation-in-part of application No. 11/597,400, filed on Nov. 22, 2006.
(60) Provisional application No. 60/573,795, filed on May 24, 2004.
(60) Provisional application No. 61/465,861, filed on Mar. 25, 2011.

(51) Int. Cl. A61M 25/14; A61D 7/00; A61M 1/00

(52) U.S. Cl. 604/28

(57) ABSTRACT

A method of treating a mammal presenting with an otic infection attributable to a pathogenic bacteria is accomplished by passing a hollow shaft having a first and second end through the tympanum of the mammal. The first end is sufficient to pierce the tympanic membrane, but flexible enough such that it buckles under the force necessary to damage any surrounding tissues in the tympanic cavity. The first end of the hollow shaft, after it pierces the tympanic membrane, extends into the tympanic cavity to aspirate a volume of effluence contained therein. The aspirated effluence is expelled out the second end of the hollow shaft. A volume of medicament (less than the volume of effluence withdrawn) is then injected through the same hollow shaft and into the tympanic cavity to treat the infection.

This application claims the benefit of the priority of U.S. Provisional Patent Application Ser. No. 61/465,861, filed on 29 Mar. 2011, and is a continuation-in-part application to U.S. patent application Ser. No. 11/597,400 filed 22 Nov. 2006, which is a national stage application of PCT/US05/18422, filed May 24, 2005 which claims the benefit of U.S. Provisional Application Ser. No. 60/573,795, filed May 24, 2004, the contents of which are incorporated in this application by reference.

### BACKGROUND OF THE INVENTION

Otitis externa involving the ear canal portion of the external ear is a common otologic problem. In the incipient stage, symptoms include itching and pain in the ear canal, and tenderness when pressure is applied around the external auditory meatus, when the ear lobe is pulled, or when the jaw is moved. In the definitive stage, suppuration occurs in the ear canal and hearing may be decreased. Over 90% of cases of otitis externa are due to bacterial or fungal infections.

Otitis media, is a term used to describe infections of the middle ear. A relatively high percentage of the population, both adults and particularly children, are affected. It has been estimated that nearly 95% of all children experience one or more episodes of otitis media by age 9, and that about 15% of all visits by children to pediatricians are in regard to otitis media. In children, the disease is most often associated with upper respiratory afflictions, which trigger a transudate secretion response in the Eustachian tube and middle ear. Bacteria and viruses migrate from the naso-pharynx to the middle ear via the Eustachian tube, and can cause the Eustachian tube to become blocked, preventing ventilation and drainage of the middle ear.

In its more severe forms, purulent exudate toxins and endogenous anti-microbial enzymes are formed in the middle ear, which can cause irreparable damage to sensory-neural and sound conducting structures. (Purulent or suppurative exudate consists of plasma with both active and dead neutrophils, fibrinogen, and necrotic parenchymal cells. This kind of exudate is consistent with more severe infections, and is commonly referred to as pus.) It has been reported that sensory-neural hearing loss occurred in 35.8% of children with otitis media with purulent exudate.

In order to treat ear disorders, it has been necessary to deliver therapeutic agents to various ear tissues in a controlled, safe, and efficient manner. For example, a variety of apparatus have been developed which are capable of delivering/administering therapeutic agents into the external auditory canal of the outer ear. For example, U.S. Pat. No. 4,034,759 to Haerr discloses a hollow, cylindrical tube manufactured of sponge material (e.g. dehydrated cellulose) which is inserted into the external auditory canal of a patient. When liquid medicines are placed in contact with the tube, it correspondingly expands against the walls of the auditory canal. As a result, accidental removal of the tube is prevented. Furthermore, medicine materials absorbed by the tube are maintained in contact with the walls of the external auditory canal for treatment purposes. Other absorbent devices designed for treatment of the external auditory canal and related tissue structures are disclosed in U.S. Pat. No. 3,528,419 to Joechle, U.S. Pat. No. 4,159,719 to Haerr, and U.S. Pat. No. 2,642,065 Negri. The Negri patent specifically describes a medicine delivery device with an internally-mounted, frangible medicine container which, when broken, releases liquid medicines into an absorbent member.

However, the delivery of therapeutic agents in a controlled and effective manner is considerably more difficult with respect the inner ear (e.g. those portions of the ear surrounded by the otic capsule bone and contained within the temporal bone which is the most dense bone tissue in the entire human body). Exemplary inner ear tissue structures of primary importance for treatment purposes include but are not limited to the cochlea, the endolymphatic sac/duct, the vestibular labyrinth, and all of the compartments (and connecting tubes) which include these components.

The inner ear tissues and regions listed above are of minimal size and only readily accessible through microsurgical procedures. In order to treat various diseases and conditions associated with inner ear tissue, the delivery of medicines to such structures is often of primary importance. Likewise, treatment of inner ear tissues and/or fluid cavities may involve altering the pressure, volume, electrical activity, and temperature characteristics thereof. Specifically, a precise balance must be maintained with respect to the pressure of various fluids within the inner ear and its associated compartments. Imbalances in the pressure and volume levels of such fluids can cause various problems, including but not limited to conditions known as endolymphatic hypertension, perilymphatic hypertension, perilymphatic fistula, intracochlear fistula, Meniere's disease, tinnitus, vertigo, hearing loss related to hair cell or ganglion cell damage/malfunction, and ruptures in various membrane structures within the ear.

Current methods of treatment generally involve the systemic use of antibiotics, and in more chronic cases the insertion of a tympanostomy tube. Topical eardrops are also used where tympanostomy tubes are present, or where the tympanic membrane has ruptured. In addition to systemic antibiotics, animals sometimes treated through an intratympanic perfusion of drugs via an incision in the tympanum

Systemic administration of antibiotics generally requires high initial doses, offers no immediate release of discomfort, and has an appreciable time lag to achieve therapeutic levels. In addition, the use of oral antibiotics to treat otic infections in children has limited efficacy, and creates a serious risk of pathogen resistance to the orally administered antibiotics.

Eardrops are typically given for infections of the outer ear and ear canal, i.e., otis externa, and offer no immediate release of discomfort. Exemplary eardrop compositions include quinolone antibiotics. For example, a topical otic composition containing the quinolone ciprofloxacin is marketed by Alcon Laboratories, Inc. under the name CILOXAN® (Ciprofloxacin 0.3%) Ophthalmic Solution, and a topical otic composition containing a combination of ciprofloxacin and hydrocortisone is marketed by Alcon Laboratories, Inc. under the name CIPRO® HC. The following quinolones have also been utilized in ophthalmic antibiotic compositions: Quinolone Product Manufacturer Ofloxacin OCUFLOX®, Allergan Norfloxacin CHIBROXIN®, Merck Lomefloxacin LOMEFLOX®, and Senju Ofloxacin has also been utilized to treat otic infections.

Intratympanic delivery of drugs has been accomplished in the past principally by making a small incision in the anesthetized tympanum and applying the drug in liquid form, allowing it to be absorbed into the tympanic cavity. Other methods include implanting a window in the tympanum, such as tympanostomy tube. A tympanostomy tube (also known as a grommet or ear tube) is a small tube inserted into the eardrum in order to keep the middle ear aerated for a prolonged period of time, and to prevent the accumulation of mucus in the middle ear. The operation to insert the tube involves a myringotomy. The tube itself is made in a variety of designs. The most commonly used type is shaped like a grommet or T. This has the disadvantage that infectious debris can be carried into the middle ear from the external canal, with the risk of creating a middle ear infection, and passage of the liquid drug into the middle ear is inhibited by the surface tension of the liquid.

Apparatus for the delivery of therapeutic agents to the middle ear, inner ear, and middle-inner ear interface tissue structures are described in U.S. Pat. Nos. 5,421,818; 5,474,529, and 5,476,446 all to Arenberg. Each of these patents discloses a medical treatment apparatus designed to deliver fluid materials to internal ear structures. U.S. Pat. No. 5,421,818 describes a treatment system which includes a tubular stem attached to a reservoir portion with an internal cavity designed to retain a supply of therapeutic fluid compositions therein. The side wall of the reservoir portion further comprises fluid transfer means (e.g. pores or a semi-permeable membrane). Contact between the fluid transfer means and the round window membrane in a patient allows fluid materials to be delivered on-demand to the round window membrane, followed by diffusion of the fluid materials through the membrane into the inner ear. U.S. Pat. No. 5,474,529 involves a therapeutic treatment apparatus with a plurality of reservoir portions (e.g. a first and a second reservoir portion in a preferred embodiment) which are connected to multiple tubular stems that are designed for implantation into the endolymphatic sac and duct using standard microsurgical techniques. Finally, U.S. Pat. No. 5,476,446 discloses a therapeutic treatment apparatus which includes a reservoir portion for retaining liquid medicine materials therein, a first tubular stem on one side of the reservoir portion, and a second tubular stem on the opposite side of the reservoir portion. The second stem is designed to reside within the external auditory canal of a patient lateral to the ear drum, while the first stem is sired for placement within an opening formed in the stapes footplate/annular ligament so that medicine materials in fluid form can be delivered into the inner ear from the reservoir portion (which resides in the middle ear cavity medial to the ear drum).

The present invention is concerned with providing a method of treating middle ear infections by (a) piercing the tympanum; (b) aspirating fluid from the behind the tympanum and (c) administering a dose of an antimicrobial compound using a compact handheld apparatus.

### SUMMARY OF THE INVENTION

Accordingly, one aspect of the present invention is directed to an apparatus which is capable of rapidly (a) piercing the tympanum; (b) evacuating the fluid contents; and (c) administering a medicament to treat any pathology behind the tympanum.

Another aspect of the invention is related to a method of treating a patient (human or animal) presenting with bacterial otitis media where fluid has collected in the tympanum, but the membrane has not perforated or ruptured. The method includes the step of passing a hollow shaft having a first and second end through the tympanum whereby the first end extends into the tympanic cavity. The method also includes the step of withdrawing a volume of purulence contained within the tympanic cavity through the first end of the hollow shaft. The method further includes expelling the withdrawn purulence out the second end of the hollow shaft. Yet additionally, the method includes administering a volume of an antibiotic solution through the hollow shaft, whereby the volume of antibiotic is administered directly into the tympanic cavity.

In another aspect, the invention is related to a dosing regime for the treatment of bacterial otitis media in an animal or a human that does not have tympanostomy tubes or a ruptured and/or perforated eardrum. The dosing regime involves administering a bactericidal quinolone antibiotic at 500 times the minimum bactericidal concentration directly into the tympanic cavity at a frequency of not more than once over a 24 hr period. The dosing regime first includes evacuating a volume of fluid or purulence from the tympanic cavity or middle ear, and then, administering a volume of the bactericidal quinolone antibiotic that is less than the volume of evacuated purulence.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a cutaway view of an exemplary apparatus capable of performing a method of the invention which shows one side of the cover removed;

FIG. 2 is a partially exploded view of the apparatus of FIG. 1;

FIG. 3 is a rear perspective view of some of the operating parts that fit within the internal portion of the barrel shown in FIG. 2;

FIG. 4 is a front perspective view of some of the operating parts that fit within the internal portion of the barrel shown in FIG. 2;

FIG. 5 is a partially exploded view of the end of the barrel assembly of the apparatus of FIG. 1;

FIG. 6 is an exploded perspective view of the barrel and turret assembly which shows a sealed antibiotic chamber and a device for opening said antibiotic chamber of he exemplary apparatus of FIG. 1;

### DETAILED DESCRIPTION OF THE INVENTION

Children are more likely than adults to suffer from otitis media. A child's eustachian tubes are smaller and more level than they are in adults. This makes it difficult for fluid to drain out of the ear, even under normal conditions. If the eustachian tubes are swollen or blocked with mucus due to a cold or other respiratory illness, fluid may not be able to drain. The present invention is directed to treating otitis media in mammals, such as a human patient 12 years or older that does not have tympanostomy tubes or a ruptured and/or perforated tympanum, or a patient between 1 and 12 years of age that does not have tympanostomy tubes or a ruptured and/or perforated tympanum, or a patient less than 1 year of age that does not have tympanostomy tubes or a ruptured and/or perforated tympanum.

The present invention is also directed at treating other mammals, such as animals. Otitis media in animals is seen in all species, but is most common in dogs, cats, horses and rabbits. Extension of infection through the auditory tube occurs in dogs, cats, and pigs. Otitis media may lead to otitis interna and inflammation of the inner ear structures. This can in turn lead to loss of equilibrium and deafness. An animal with otitis media may circle and fall toward the affected side and will have generalized uncoordination that may be severe enough to cause difficulty in rising and ambulating. Fluid present in the tympanic cavity or sclerotic changes of the osseous tympanic bullae may be detected radiographically. Also, it is generally recommended that cytologic examination (Gram's stain and Wright's stain) and culture of the exudate/purulence be taken. In animals with otitis media where the tympanum is bulging or discolored, acceptable veterinary practice is to perforate the tympanum (perform a myringotomy) to permit culture of the fluid, to relieve the pressure (and thus the pain) within the middle ear, and to permit removal of the inflammatory exudate. However, perforation of the tympanum could result in permanent diminished hearing loss. See The Merck Veterinary Manual, 2008, Merck & Co., Inc., Whitehouse Station, N.J. USA. Accordingly, one aspect of the present invention is directed to treating an animal such as a canine, feline, equine, porcine, or rabbit presenting with otitis media. This application incorporates by reference the text of application Ser. No. 11/587,400, filed Nov. 22, 2006 from page 6, line 24 to page 13, line 15.

Otitis media has many degrees of severity, and various terms are used to describe the condition. As used herein, otitis media includes acute otitis media, acute bacterial otitis media, viral acute otitis media, serous or secretory otitis media (otorrhea), and otitis media with effusion. Acute otitis media may also be viral and as such is self-limited.

There is congestion of the ears and mild discomfort and popping. If the middle ear, which is normally sterile, becomes contaminated with bacteria, pus and pressure in the middle ear can result. This is called acute bacterial otitis media. Viral acute otitis media can lead to bacterial otitis media in a very short time. The individual with bacterial acute otitis media has the classic “earache” pain that is more severe and continuous. More severe bacterial cases may result in perforation of the ear drum, and the infection of the mastoid space (mastoiditis).

Otitis media with effusion, also called serous or secretory otitis media, is simply a collection of fluid, or effluent, that occurs within the middle ear space as a result of the negative pressure produced by altered Eustachian tube function. This can occur purely from a viral upper respiratory infection, or bacterial infection, or it can precede and/or follow acute bacterial otitis media. There may be enough purulence (i.e., pus) that it drains to the outside of the ear (otorrhea), or the purulence may be minimal enough to only be seen on examination using a binocular microscope. Over weeks and months, middle ear fluid can become very thick and glue-like which increases the likelihood of its causing conductive hearing impairment.

In one aspect, the present invention is directed to a method for treating otitis media in a patient presenting with a collection of fluid, serous, effluent, or pus, behind the tympanum, where the tympanum has not perforated or ruptured.

In another aspect, the present invention is directed to a method of treating a mammal presenting with bacterial otitis media. Known pathogenic bacterial species responsible for ottis media include, but are not limited to, Streptococcus pneumoniae, Streptococcus pyogenes, non-typeable or type b Haemophilus influenzae, Moraxella catarrhalis, coagulase-negative staphylococci, anaerobic bacteria, gram-negative bacilli and Staphylococcus aureus (found more commonly in neonates presenting with otitis media).

In yet another aspect, the method of the present invention involves administration of a medicament, such as an antibiotic, to treat the bacterial otitis media. Antibiotics are categorized as bactericidal if they kill the susceptible bacteria or bacteriostatic if they reversibly inhibit the growth of bacteria. Exemplary antibiotics of the present invention are bactericidal, and include quinolones, such as, but not limited to, ofloxacin, difloxacin, enrofloxacin, ciprofloxacin, ibaflaxacin, marbofloxacin, and orbifloxacin and their pharmaceutically acceptable salts, e.g., the hydrochloride salt. In an exemplary embodiment of the invention, the antibiotic is 0.3% ofloxacin solution by weight.

Other types of non-antibiotic medicines that have been used to treat inner ear conditions include but are not limited to urea, mannitol, sorbitol, glycerol, lidocaine, xylocaine, epinephrine, immunoglobulins, sodium chloride, steroids, heparin, hyaluronidase, antioxidants, neurotrophins, nerve growth factors, various therapeutic peptides, and polysaccharides.

An exemplary antibiotic of the present invention is in solution or suspension form having a concentration of 0.1, 0.2, 0.3, 0.4, 0.5, 0.6, 0.7, 0.8, 0.9, 1, 2, 3, 4, and 5% or 0.1 to 5 by weight based on the weight of the solution or suspension. Alternatively, the antibiotic solution of the invention has a concentration in the range selected from the group consisting of 0.1 to 5, 0.2 to 4, 0.5 to 3, 0.75 to 2, and 0.95 to 1.0% or 0.1 to 5% by weight of the solution or suspension.

The medicament, whether a small molecule medicament, protein or antibiotic, is, in one embodiment, formulated to release in a controlled manner, where once the medicament is injected into the tympanic cavity, it releases the medicament over a period of time. For example over 2, 4, 6, 8, 12, 14, 16, 18, 24, 30, 36, 42, 48, 60, 72, 84, 96, 120, 144, or 168 hrs. Exemplary controlled release formulations are formulated via prodrugs and various blends of excipients, such as the technology described in US Published Patent Application 2010/0291220, herein incorporated by reference.

According to an aspect of the present invention, the antibiotic is administered into the tympanic cavity at a concentration that is greater than 10, 50, 100, 150, 200, 250, 300, 350, 400, 450, or 500 or 10-500, or 50-400 or 100 to 300 times the minimum bactericidal concentration that is necessary to kill the pathogenic bacterial species responsible for the infection. The minimum bactericidal concentration (MBC) is the lowest concentration of the antibiotic that kills 99.9% of the original inoculum in a given time. For any antibiotic to be effective, the MBC must be achieved at the site of the infection. In the case of otitis media, that location is in the tympanic cavity.

The antibiotic solution of the invention is administered to the tympanic cavity at a volume in the range of 0.2 to 5, 0.5 to 4, 0.75 to 3, and 1 to 2 mL. Alternatively, the volume of antibiotic administered to the tympanic cavity is 0.2, 0.5, 0.75, 1, 1.5, 2, 3, 4 and 5 mL.

If the otitis media has progressed to serous or purulent otitis media, recognized by inflammation of the middle ear in which there is fluid accompanied a bulging eardrum, injecting an antibiotic solution into the tympanic cavity may increase the pressure behind the tympanum. The increased pressure may cause an increase in the discomfort of the patient and potentially rupture the tympanum. According to an aspect of the invention, a flexible needle penetrates the tympanum and protrudes into the tympanic cavity, a volume of fluid, pus, exudate, purulence (herein referred to as “effluent”) may be drawn up or aspirated through the hollow shaft and expelled out the second end of the hollow shaft. The volume of effluence contained within the tympanic cavity may be aspirated through the hollow shaft via vacuum. In an aspect of the invention, once the hollow shaft is inserted through the tympanum and extends into the tympanic cavity, the hollow shaft remains inserted through the tympanum during the steps of aspirating the effluent and injecting the medicament. In this way, the fragile, already inflamed and stressed tympanum need not be punctured more than once.

The volume of effluent aspirated from the tympanic cavity is more than 0.25, 0.5, 0.75, 1.0, 1.25, 1.5, 1.75, 2.0, 2.5, 3.0 and 3.5 ml or 0.25-3.5 ml; 0.5-3.0 ml or 0.76-2.5 ml or 1.0-2.0 ml depending on the size of the patient, whether the patient is a human infant, child or adult, or whether the patient is a dog, cat, or horse. In an embodiment of the invention, the volume of effluent aspirated is equal to or greater than the volume of the medicament injected. In one aspect, the volume of medicament injected is 5, 10, 15, 20, 25, 30, 35, 40, 45, 50, 55, 60, 65, 70, 75, 80, 85, 90, 95, 100 percent of the volume of effluent aspirated. The volume is between 5 and 100% based on the total estimated volume of effluent present in the middle ear. The volume aspriated should be less than the volume injected so as to affect a decrease in pressure on the tympanum.

In another embodiment, the invention is related to a dosing regime for the treatment of bacterial otitis media. Prior to injecting a volume of medicament, such as a bactericidal quinolone antibiotic, into the tympanic cavity to reach 500 times the minimum bactericidal concentration, a volume of effluent is aspirated from the tympanic cavity. The dose required to reach 500 times the minimum bactericidal concentration may be determined as follows: One ml of a 0.3% by weight solution or suspension of a floxin otic preparation, based on the total weight of the solution or suspension will deliver approximately 3000 mcg of active drug to the middle ear, The volume capacity of the middle ear is between approximately 1 and 5 m. MIC (minimum inhibitory concentration) are typically below about 2 mcg/ml for a range of microorganisms. This suggests that the MIC of such a dose will be upwards of 300 times the MIC. The concentration of the injectable solution or suspension is 0.1 to 5% by weight based on total weight of the solution or suspension. Other ranges include from 0.2 to 4%; 0.5 to 3%; 0.75 to 2%; 0.95 to 1% (all percents are by weight based on the total weight of the solution or suspension.

The preferred volume of the medicament solution or suspension injected into the tympanic cavity will be between 0.1 and 2 ml of medicament solution or suspension; or 0.1-2 ml; or 0.1 and 0.75 ml; 0.1 and 0.5 ml or 0.1 ml and 0.25 ml.

The preferred volume of the medicament solution or suspension injected into the tympanic cavity may also be between 0.2 and 5 ml; 0.2 and 4 ml; 0.2 and 3 ml; 0.2 and 2 ml; 0.2 and 1.5 ml; 0.2 and 1 ml; 0.2 and 0.75 ml.

These exemplary steps of the method of the present invention should be administered at a frequency of not more than once over a 24, 48, 72, 96, or 120 hr period or a period of 24 to 120 hours. In an exemplary embodiment, the method of the present invention, after 1 or 2 treatments in a 48 hour period, results in greater than 75%, 80%, 85%, 90%, 95%, or 99% of the Microbacterium species being destroyed within 3 days after treatment.

### EXAMPLE

The following example illustrates an exemplary device that allows a skilled practitioner to carry out an exemplary method of the invention described herein.

### Example 1

According to an aspect of the method of the invention, the antibiotic solution is administered directly into the tympanic cavity by passing a hollow shaft having a first and second end through the tympanum whereby the first end extends into the tympanic cavity. In certain embodiments of the invention, the hollow piercing shaft is a single or double bore, medical grade, stainless steel needle or polyamide-type flexible cannula. In one embodiment, the step of passing the hollow piercing shaft through the tympanum is actuated by a spring-loaded mechanism of an apparatus of the invention or an operator.

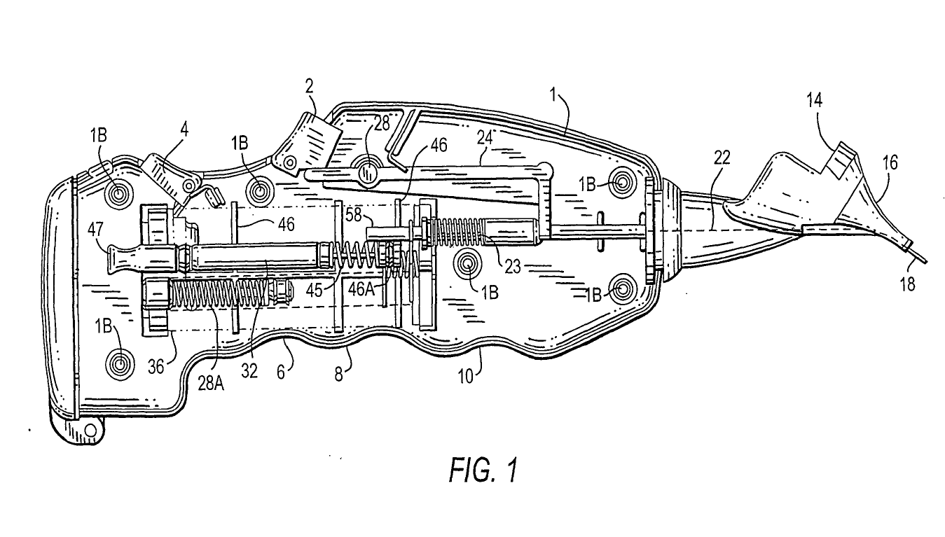

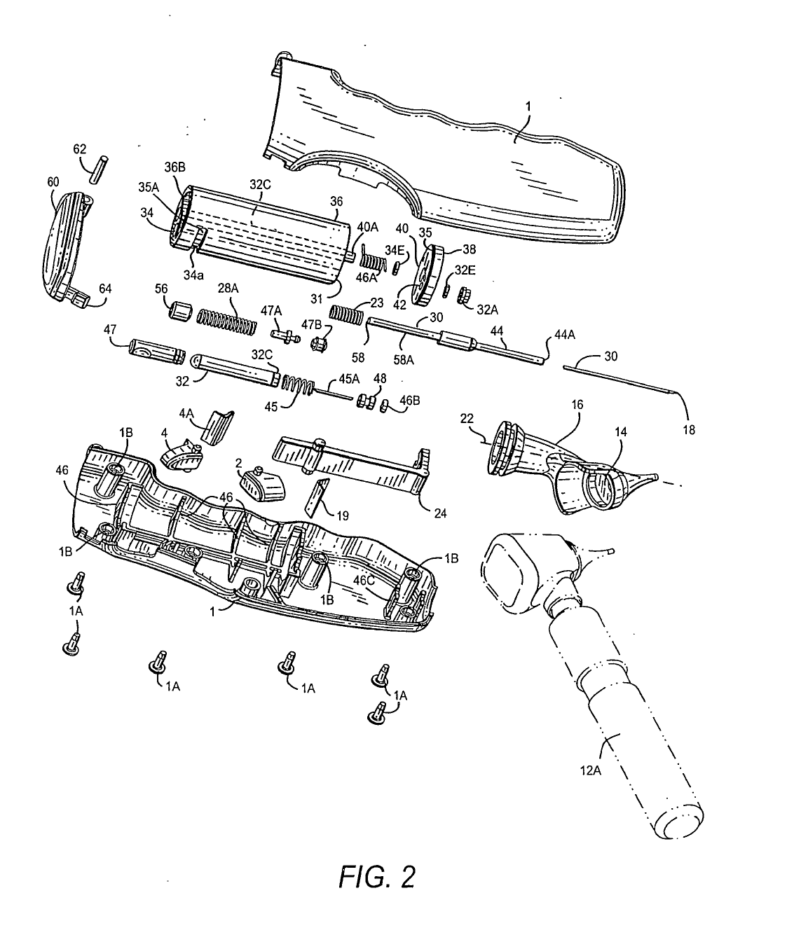

As best seen in FIG. 1, an exemplary apparatus to carry out an exemplary method of the invention comprises an external cover 1 having two halves held together by rivets placed in rivet holes 1B. A first actuating trigger 2 and a second actuating trigger 4 are mounted on the cover. The cover 1 is provided with a plurality of indentations 6, 8 and 10 that serve as ergonomically shaped finger gripping surfaces. At the forward end 12 of the apparatus, a viewing opening 14 is provided that is adapted to engage a standard otoscope 12 (in FIG. 2). At the tip 14 of the viewing cannula 16, the needle 18 is shown in a partially extended position as if it was being extended for the purpose of piercing the tympanum.

The tympanic cavity consists of two parts: the tympanic cavity proper, opposite the tympanum; and the attic or epitympanic recess, above the level of the membrane. The latter contains the upper half of the malleus and the greater part of the incus. Including the attic, the vertical and antero-posterior diameters of the cavity are each about 15 mm. The transverse diameter measures about 6 mm above and 4 mm below; opposite the center of the tympanum, it is only about 2 mm. The tympanic cavity is bounded laterally by the tympanum; medially, by the lateral wall of the internal ear; it communicates, behind, with the tympanic antrum and through it with the mastoid air cells, and in front with the auditory tube. According to another aspect of the invention, the hollow piercing shaft is inserted through the tympanum and extends beyond the tympanum into the tympanic cavity at a distance of not more than a 6, 5.5, 5, 4.5, 4, 3.5, 3,2.5, 2, 1.5, or 1 mm.

The viewing opening 14 is preferably arranged so that it allows the physician to view the tympanum as the needle 18 pierces the tympanum. As shown in FIG. 1, the partially extended needle 18 follows the path shown by dotted line 22 as it is extended by the spring 23 which is controlled by latch 24 that pivots at mounting pivot 28 when first actuating trigger 2 is depressed. Spring band 19 provides a resistance that holds trigger 2 in the open position. Needle 18 is preferably made of a resilient material such as a polyimide that is flexible enough to bend as it is passed through an internal channel shown by dotted line 22 in viewing cannula 16. The needle cartridge assembly 30, vacuum cartridge 32 and medicament chamber 34 are shown within barrel 36 which is shown within cover 1 by dotted lines. Barrel 36 is a cylindrical structure provided with a space 36B that receives the needle cartridge assembly 30 and separate chambers for the vacuum cartridge 32 and the medicament chamber 34.

The barrel 36 comprises a cylindrical structure which is divided into a hollow first chamber 36B and an offset core of solid material that has two separate chambers. The first chamber acts as a mounting means for vacuum cartridge 32 and the second chamber is medicament chamber 34.

Dotted lines 34C on barrel 36 show the position of medicament chamber 34 that houses the medicament and its associated delivery elements which comprise a spring, a plunger element and a seal which engages end 58 of needle retainer element 44A when barrel 36 is rotated into position for ejecting medicament. Dotted lines 32C show the position in barrel 36 where vacuum cartridge 32 is placed. End cap 60, which allows access to the inside of the cover 1 is mounted at the end of cover 1 opposite the end on which tip 14 is mounted, on a movable axle 62 and is held in a closed position with locking tab 64.

As best seen in FIG. 2, a partially exploded view of the otic aspirator/dispenser of FIG. 1 is disclosed which has a view of otoscope 12A which may be placed in opening 14 of cannula 16. At the forward end 31 of the barrel 36 is a turret 38 which has an axially located opening 40 and remains fixed while the barrel 36 is rotated from a first position where the needle assembly is in register with an off center hole 42 in turret 38 that allows the needle to be extended from the barrel by the action of spring 23. The barrel 36 is mounted within cover 1 on complimentary molded half circle shaped supports 46 which are on the inside of both cover halves but are only shown on one side of cover 1 in FIG. 2.

The forward end 31 of the barrel 36 has an opening into space 36B and is also provided with an axle 40A, which is fitted into hole 40 in turret 38. Opening 30B is provided to allow the shaft 58A below needle retainer element 44A to move out of the barrel 36 when trigger 2 is activated. Opening 32D is provided to allow vacuum cartridge 32 to communicate with the end 58 of needle cartridge assembly 30 when barrel 36 is rotated by spring 46A. O-ring 32E is placed in recess 32F to provide a seal between opening 32D and hole 42. Opening 34D is provided to allow medicament chamber 34B to communicate with the end of needle cartridge assembly 58 when barrel 36 is rotated by spring 46A when trigger 4 is activated. O-ring 34E is held in recess 34F to seal the medicament chamber and the end 58 of needle assembly 30 when the barrel 36 rotates to allow opening 34D to communicate with the end 58 of needle assembly 30.

Spring 23 is released by the action of lever 24 which is controlled by trigger 2. As the needle cartridge is extended by the action of spring 23, the end 58 of needle retainer element 44 is extended past the end of barrel 36 which allows barrel 36 to be axially rotated by the action of an axial spring 46A to cause vacuum cartridge 32 to move into register with off center hole 42 which is in communication with needle 18. As the vacuum cartridge tube 32 rotates into register with off center hole 42, a detent 35A in groove 34A stops rotary movement of the barrel 36. The vacuum cartridge 32 is opened to place a vacuum on needle 18 by opening end cap 60 and digitally depressing plunger element 47 against the force exerted by spring 45 to cause needle 45A to break the seal 32A on the vacuum in vacuum cartridge 32. The effect of the vacuum on the end of needle 18 is to aspirate any fluid in the tympanic cavity or middle ear.

The vacuum cartridge 32 and the medicament chamber 34 are within barrel 36. The vacuum cartridge is preferably provided with a vacuum which may be approximately P<5×10−3 Pa.

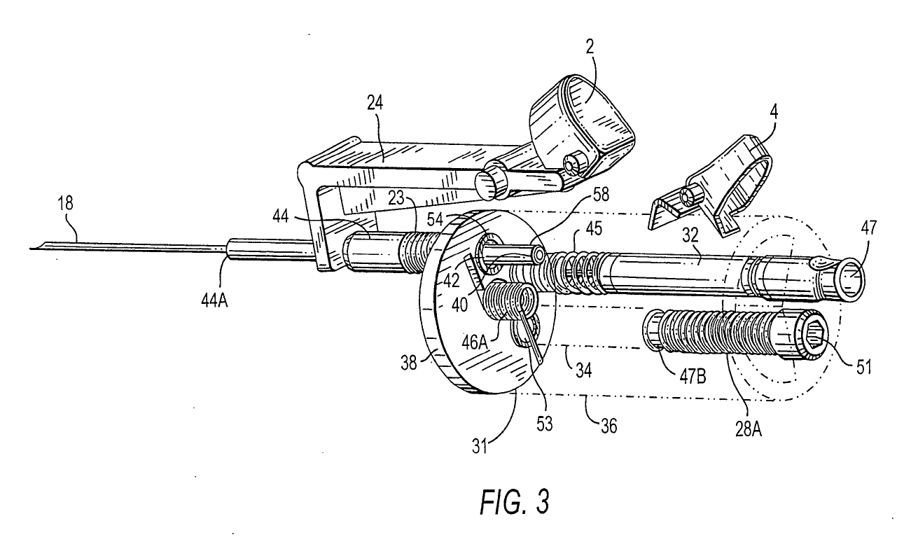

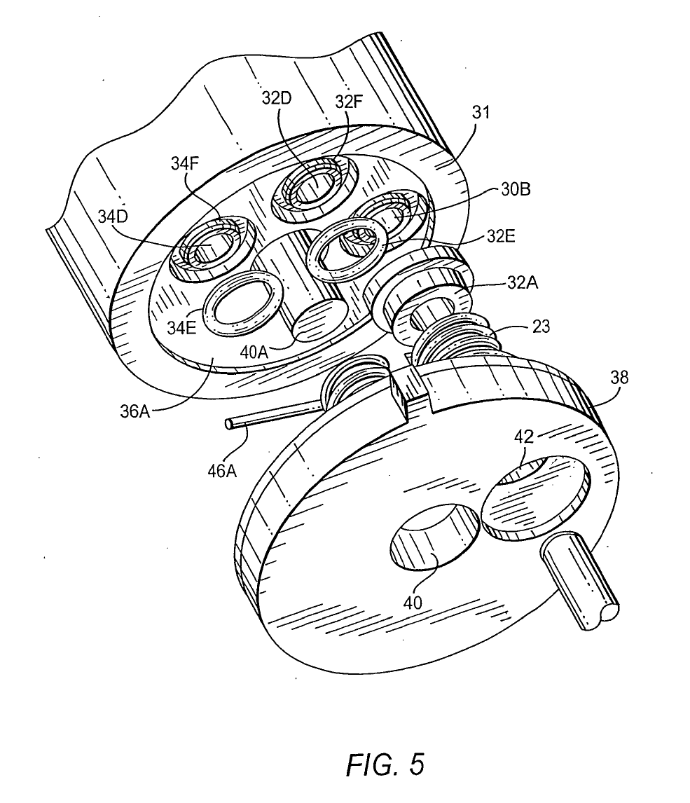

As best seen in FIG. 5, turret 38 is provided with axial hole 40 and off center hole 42. The barrel 36 rotates due to the action of axial spring 46A to bring the vacuum cartridge 32 into register with off center hole 42. The vacuum cartridge 32 is provided with a constricted end portion 48. This constricted end portion 48 engages off center hole 42 when the vacuum cartridge contacts the off center hole 42A. A small needle 45A in constricted end portion 48 which is mounted at the end of vacuum tube 32 breaks the vacuum seal 32C as plunger element 47 is manually depressed. O-ring 46B is mounted inside of barrel 36 to seal the end of vacuum cartridge 32 against the inside of opening 32D. Spring 45 is held in recess 54 as shown in FIG. 3 on the inner side of offset hole 42 so that it does not move out of alignment with offset hole 42. The end 58 of needle retainer element 44 is adapted to rotate so that it is centered on O-ring 32E in back of spring retainer 32A. Needle 18 is attached to the end of needle retainer element 44A, which forms a conduit from the tip of needle 18 to end 58. Expanded mid-section of needle retainer element 44 cooperates with retainer stop 46C to prevent end of needle retainer element 58 from being extended beyond the end of turret 38 by the action of spring 23. Thus, in operation, as needle cartridge assembly 30 is extended from barrel 36 by the action of spring 23, the action of the axial spring 46A on barrel 36 causes the barrel 36 to rotate until it reaches a detent 35A set in groove 34A where trigger 4 extension 4A causes the rotary movement of the barrel to stop and which causes the vacuum tube 32 to be held in register with offset hole 42. As described above, digital pressure is used to break the vacuum seal with needle 45A to cause the seal on said vacuum cartridge 32 to open and expose the tympanic cavity or middle ear to the vacuum in the vacuum cartridge which is sufficient to extract any fluid from behind the tympanum through needle 18 without damaging any of the inner ear tissues.

As best seen in FIG. 3, medicament chamber 34 is shown by dotted lines within barrel 36. The medicament chamber 34 is provided with a constricted end portion which acts as seal 47B.

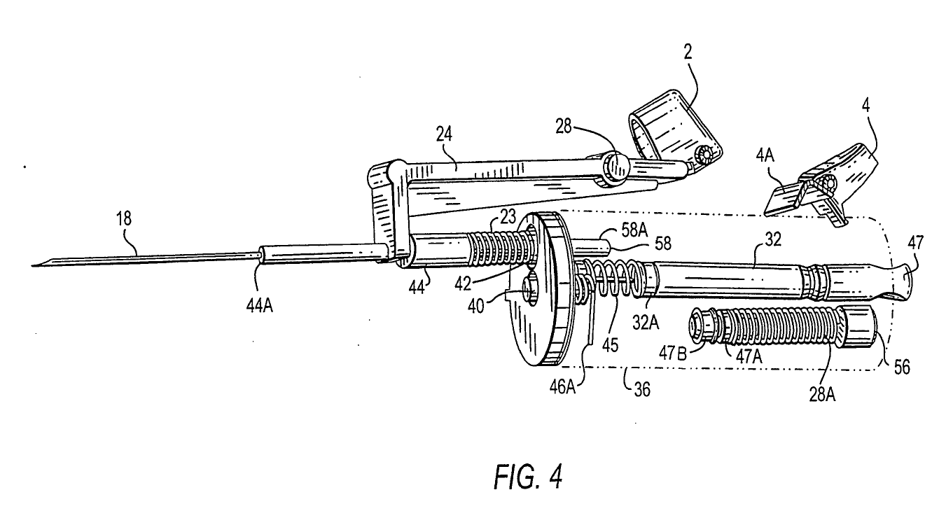

As best seen in FIG. 4, spring retainer element 56 is positioned against spring 28A which contacts connector element 47A which contacts seal 47B in medicament chamber 34 in barrel 36. The medicament is stored in medicament chamber 34 between seal 47B and turret 38. When trigger 4 is activated, detent 35A is bypassed and barrel 36 rotates to allow medicament to be ejected, through needle 18 by the action of spring 28A on connector 47A and seal 47B, into the space behind the tympanum.

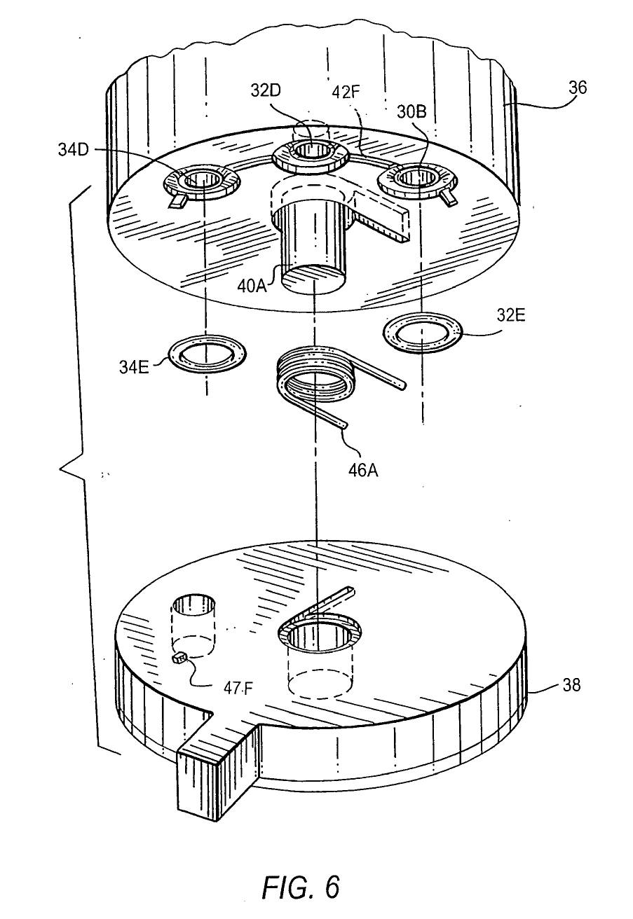

As best shown in FIG. 6, the end of seal 47B in medicament chamber 34 may be ruptured by cutting element 47F on turret 36 as the barrel 38 rotates and passes over the end of the medicament chamber 34 under the action of a spring which is released by the trigger. Recessed channel 42F is provided in barrel 36 to allow cutting element 47F to move across the vacuum chamber and over the medicament chamber where it breaks seal 47B.

After medicament is injected into the middle ear and the device is removed from the patient's ear, vacuum cartridge 32 can be removed from barrel 36 for testing of effluent aspirated from the tympanic cavity or middle ear.

According to this exemplary apparatus for carrying out an exemplary method of the invention, the apparatus includes the needle cartridge, vacuum cartridge and medicament chamber are all manufactured so that they are sterile when delivered to the physician. Various procedures are well known for the preparation of pre-filled sterile applicators and for packaging these devices in suitable containers to maintain sterility.

What is claimed:

1. A method of treating a mammal presenting with an otic infection attributable to a pathogenic bacteria, the method comprising the steps of:
(i) passing a hollow shaft having a first and second end through the tympanum of the mammal whereby the first end extends into the tympanic cavity;
(ii) aspirating a volume of effluence contained within the tympanic cavity through the first end of the hollow shaft;
(iii) expelling the withdrawn effluence out the second end of the hollow shaft; and
(iv) injecting a volume of a medicament through the hollow shaft, whereby the volume of medicament is injected into the tympanic cavity.

2. The method of claim 1, wherein the medicament is a bactericidal antibiotic.

3. The method of claim 1, wherein 75% to 100% of the pathogenic bacteria are destroyed within 3 days after completing a single application of steps (i) though (iv).

4. The method of claim 1, wherein the step of passing the hollow shaft through the tympanic membrane is actuated by a spring-loaded mechanism or an operator of an apparatus.

5. The method of claim 1, wherein a distance the hollow shaft travels after passing though the tympanum is limited to 6 mm or less.

6. The method of claim 1, wherein the hollow shaft is a single or double bore stainless steel needle or flexible cannula.

7. The method of claim 1, wherein the step of aspirating the volume of effluence is through application of a vacuum.

8. The method of claim 1, wherein the volume of effluence withdrawn is greater than the volume of the medicament solution injected into the tympanic cavity.

9. The method of claim 1, wherein the volume of the medicament solution or suspension injected into the tympanic cavity is equal to 5-100% of the volume of aspirated effluent.

10. The method of claim 1, wherein the volume of the medicament solution or suspension injected into the tympanic cavity is in the range of 0.2 to 5 mL.

11. The method of claim 1, wherein the volume of the medicament solution or suspension injected into the tympanic cavity is from 0.2 to 5 mL.

12. The method of claim 1, wherein the medicament is a bactericidal antibiotic solution or suspension has a concentration in the range of 0.1 to 5% by weight of the total weight of solution or suspension.

13. The method of claim 1, wherein the medicament is a bactericidal antibiotic solution or suspension having a concentration selected from the group consisting of 0.1 and 5% by weight of the total weight of solution or suspension.

14. The method of claim 1, wherein the medicament is a bactericidal antibiotic solution or suspension and is injected into the tympanic cavity to provide a concentration of the medicament that is greater than 10 to 500 times the minimum inhibitory concentration that is necessary to kill the pathogenic bacteria.

15. The method of claim 2, wherein the bactericidal antibiotic is a quinolone selected from the group consisting of ofloxacin, difloxacin, enrofloxacin, ciprofloxacin, ibaflaxacin, marbofloxacin, and orbifloxacin or salts thereof.

16. The method of claim 1, wherein the mammal is a human patient from a newborn to 12 years of age.

17. The method of claim 16, wherein the patient does not have tympanostomy tubes or a ruptured and/or perforated tympanum.

18. The method of claim 1, wherein the mammal is selected from the group consisting of canine, feline and equine.

19. A method of treating bacterial otitis media in a patient 12 years or older that does not have tympanostomy tubes or a ruptured and/or perforated tympanum, or a patient between the ages of a newborn and 12 years of age that does not have tympanostomy tubes or a ruptured and/or perforated tympanum, or a patient less than 1 year of age that does not have tympanostomy tubes or a ruptured and/or perforated tympanum, the method comprising the steps of:
aspirating a volume of effluent from within the tympanic cavity or middle ear of the patient; and
injecting a dose of a bactericidal antibiotic solution or suspension through the tympanum membrane into the tympanic cavity or middle ear in an amount that reaches at least 500 times the minimum inhibitory concentration that is necessary to kill the bacteria.

20. The method of claim 19, wherein the aspirating step evacuates a volume of effluence that is greater than 1 mL.

21. The method of claim 19, wherein the bactericidal antibiotic solution is 0.3% ofloxacin by weight based on the total weight of the solution.

22. The method of claim 19, wherein the steps of aspirating and injecting are administered not more than once over a period of 24-120 hrs.

23. A dosing regime for the treatment of bacterial otitis media in patients presenting with a non-ruptured or non-perforated tympanum and that do not have implanted tympanostomy tubes comprising administering a bactericidal quinolone antibiotic solution or suspension in an amount that is 500 times the minimum bactericidal concentration into the tympanic cavity at a frequency of not more than once over a 24 hr period by aspirating a volume of effluent from the tympanic cavity or middle ear of the patient and then injecting a volume of the bactericidal quinolone antibiotic solution or suspension that is less than the volume of aspirated effluent.

26. A dosing regime for the treatment of bacterial otitis media in a canine, feline or equine comprising administering a bactericidal quinolone antibiotic solution or suspension at 500 times the minimum bactericidal concentration into the tympanic cavity at a frequency of not more than once over a 24 hr period by aspirating a volume of effluent from the tympanic cavity or middle ear and then injecting a volume of the bactericidal quinolone antibiotic that is less than the volume of aspirated effluent.
